(19) United States
(12) Patent Application Publication
(10) Pub. No.: US 2003/0215403 A1
(43) Pub. Date: Nov. 20, 2003

(54) Oral composition

(75) Inventors: Andrew Joiner, Bebington (GB); David William Thornthwaite, Bebington (GB); Philip Christopher Waterfield, Bebington (GB)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/205,351

(22) Filed: Jul. 25, 2002

(30) Foreign Application Priority Data
Jul. 26, 2001 (EP) 01306397.9

(51) Int. Cl. A61K 7/20

(52) U.S. Cl. 424/53

(57) ABSTRACT

An oral composition with an improved teeth whitening effect, comprising a first formulation comprising an effective amount of a peroxy amido phthalamide having the structural formula:

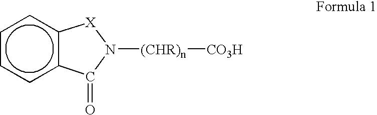

in which R is hydrogen or C₁-C₄ alkyl; n is 1 to 5; and X is C═O or SO₂,

wherein said first formulation has a pH of from 2 to 6

and a second formulation formulated such that when said second formulation is mixed with the first formulation the pH of the mixture is from 6.5 to 8.5.

### BACKGROUND TO THE INVENTION

The use of peroxy compounds in oral care compositions has already been proposed in the prior art. Many peroxy compounds have been suggested for whitening/bleaching human teeth, and representative examples of such peroxy compounds are hydrogen peroxide, urea peroxide, organic peracids such as perphthalic acid, diperoxycarboxylic acids, 1,12-dodecanedioic peroxy acid, peroxy acetic acid and systems comprising a peroxy compound and a peroxy acid precursor which generate peroxy acetic acid in situ, such as sodium perborate and tetraacetylethylene diamine (TAED). The use of peroxy acetic acid is suggested in particular in e.g. EP-A-0545,594 (Colgate), which also sets out the various prior proposals, made in the art for several peroxy compounds as bleaching/whitening agent for human teeth.

Peroxy amido phthalamides have the structural formula:

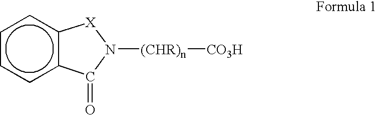

in which R is hydrogen or C₁-C₄ alkyl; n is 1 to 5; and X is C═O or SO₂, are known per se and have been described in EP-A-325,288 and EP-A-325,289. A preferred compound of this formula is N-phthalimido hexanoic peroxy acid (“PAP”) of Formula 1, in which R is H, n is 5 and X is C═O. An alternative example of a compound according to Formula 1 wherein x is SO₂ is saccharin-perhexanoic acid (“saccharin PAP”), as described in EP-A-485,927.

For the sake of clarity, the term ‘peroxy amido phthalamides’ hereinafter means those peroxy amido phthalamides according to Formula 1.

WO 00/16737 (McLaughlin) discloses the use of potassium based inorganic persalts and peroxide bleaching agents in a two component system. This system effects the whitening of teeth, but does not damage the surrounding tissue.

U.S. Pat. No. 6,165,448 A, (Chesebrough-Ponds) discloses a tooth whitening method using an organic peroxy compound in combination with an imine quaternary salt acting as a catalyst.

### SUMMARY OF INVENTION

We have now found that the provision of peroxy amido phthalamides as oral care actives can be improved when formulated in a multi-component product in which the component comprising the peroxy amido phthalamide has a pH ranging from 2 to 6 and is mixed with another formulation just prior to use such that the mixture of the two formulations has a pH ranging from 6.5 to 8.5.

Without wishing to be bound by theory we believe that peroxy amido phthalamide bleaching agents, notably PAP, have special characteristics that make them particularly suitable for use in this system. Having mixed the components so that they are within the pH range from 6.5 to 8.5, the peroxy amido phthalamide is only sparingly soluble. This property of peroxy amido phthalamide means at any one time there is only a small amount of bleaching agent in solution and thus available to bleach the substrate. Once this activated bleach has reacted it is then replaced by some more of the insoluble material. This keeps the solution concentration of the peroxy amido phthalamide at its saturation level.

The slow dissolution of the peroxy amido phthalamide in this manner extends the time period over which the peroxy amido phthalamide will be effective at bleaching.

This property of the peroxy amido phthalamide also remedies the problem of the bleaching agent not being stable within the pH range specified for the mixture, namely pH from 6.5 to 8.5. Therefore the pH of the solution can be instantaneously increased to from pH 6.5 to 8.5, (which is the optimum conditions for the bleaching agent active to work), without compromising the stability of the peroxy amido phthalamide, i.e. the stability of the peroxy amido phthalamide in a system whose pH changes is not dependent on the new pH.

### DETAILED DESCRIPTION OF INVENTION

Accordingly, the invention provides an oral composition with an improved teeth whitening effect, comprising a first formulation comprising an effective amount of a peroxy amido phthalamide having the structural formula:

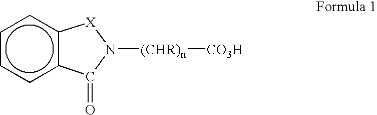

in which R is hydrogen or C₁-C₄ alkyl; n is 1 to 5; and X is C═O or SO₂,

wherein said first formulation has a pH of from 2 to 6

and a second formulation formulated such that when said second formulation is mixed with the first formulation the pH of the mixture is from 6.5 to 8.5.

Preferably, the peroxy amido phthalamide is PAP.

Preferably the second formulation has a pH ranging from 9 to 12.

More preferably, the pH of the mixture of the first formulation and the second formulation ranges from 7 to 8.

Most preferably, there is only one second formulation.

The amount of peroxy amido phthalamide, used in the present invention, may vary from 0.0001 to 99% by weight of the total oral composition, preferably from 0.001 to 30% by weight, particularly preferably from 0.01-10% by weight and especially preferably from 0.1 to 5% by weight of the total oral composition.

Further the first and second formulations of the oral care composition according to the invention are packaged independently from one another, i.e. they are stored separately and mixed only immediately prior to use. The separate packagings may be part of a greater packaging presentation which allows for two formulations to be dispensed simultaneously and mixed for the first time on the brush such as that used by Mentadent® in their so called double pump mechanisms. The packages may also be completely independent from one another and be part of a whitening kit which contains a container of bleaching active and a container of buffering agent from which the contents can be independently dispensed and mixed before use.

The composition according to the invention is meant to be used in the oral cavity and as such will comprise an oral care benefit agent selected from the group consisting of anti-caries agent, anti-tartar agent, gum-health agent, antimicrobial agent and anti-oral malodour agent. The oral care benefit agent may be in either of the formulations according to the invention, though preferably the pH adjusting formulation, i.e. the formulation not comprising the bleaching agent. Preferably the oral composition will comprise a fluoride source as anti-caries agent.

The oral composition can be formulated in any suitable application form, such as gels, mouthwashes and toothpastes. The composition may also be a so called leave-on product whereby it is dispensed into a gum shield and then applied directly to the teeth and left for several minutes before being removed.

The oral care compositions of the present invention may furthermore comprise optional, conventional ingredients such as pharmaceutically acceptable carriers like starch, sucrose, water or water/alcohol systems etc. Small amounts of surfactants may also be included, such as anionic, non-ionic and amphoteric surfactants. When formulated into a dentifrice, such formulation may contain all the usual dentifrice ingredients.

Thus, they may comprise particulate abrasive materials such as silicas, aluminas, calcium carbonates, dicalciumphosphates, calcium pyrophosphates hydroxyapatites, trimetaphosphates, insoluble hexametaphosphates and so on, usually in amounts between 5 and 60% by weight of the total oral composition.

Furthermore, the oral care composition formulations may comprise humectants such as glycerol, sorbitol, propyleneglycol, xylitol, lactitol and so on.

Binders and thickeners such as sodium carboxymethyl-cellulose, xanthan gum, gum arabic, etc. may also be included, as well as synthetic polymers such as polyvinyl pyrrolidone, Gantrez®, polyacrylates and carboxyvinyl polymers such as Carbopol®.

Flavours such as peppermint and spearmint oils may also be included, as well as preservatives, opacifying agents, colouring agents, pH-adjusting agents, sweetening agents and so on. Stabilising agents for the organic peroxy acids such as dipicolinic acid or sodium stannate may also be usefully included.

Anti-bacterial agents may also be included such as Triclosan, chlorhexidine, copper-, zinc- and stannous salts such as zinc citrate, sodium zinc citrate and stannous pyrophosphate, sanguinarine extract, metronidazole. Further examples of anti-bacterial agents are quaternary ammonium compounds such as cetylpyridinium chloride; bis-guanides such as chlorhexidine digluconate, hexetidine, octenidine, alexidine; halogenated bisphenolic compounds such as 2,2′methylenebis-(4-chloro-6-bromophenol).

Polymeric compounds which can enhance the delivery of active ingredients such as anti-bacterial agents can also be included. Examples of such polymers are copolymers of polyvinylmethylether with maleic anhydride and other similar delivery enhancing polymers, e.g. those described in DE-A-3,942,643 (Colgate).

Furthermore anti-inflammatory agents such as ibuprofen, flurbiprofen, aspirin, indomethacin etc. may also be included.

Anti-caries agents such as sodium- and stannous fluoride, aminefluorides, monosodiumfluorophosphate, casein, plaque buffers such as urea, calcium lactate, calcium glycerophosphate, strontium polyacrylates may also be included. Other optional ingredients include vitamins such as Vitamin C, and plant extracts. Desensitising agents such as potassium citrate, potassium chloride, potassium tartrate, potassium bicarbonate, potassium oxalate, potassium nitrate as well as strontium salts may also be included.

Buffers and salts to buffer the pH and ionic strength of the compositions may also be included. Liposomes and other encapsulates may also be used to improve delivery or stability of active ingredients.

Furthermore, the oral compositions may comprise anti-calculus agents such as alkalimetal pyrophosphates, hypophosphite-containing polymers, organic phosphonates, phosphocitrates etc.

In addition, the compositions may comprise functional biomolecules such as bacteriocins, antibodies, enzymes and so on.

Other optional ingredients that may be included are e.g. bleaching agents, e.g. those described in EP-A-0 545,594, effervescing systems such as sodium bicarbonate/citric acid systems, colour change systems, and so on.

When formulated as a mouthwash, the oral care composition usually comprises a water/alcohol solution, flavour, humectant, sweetener and colorant.

Since the peroxyacids of the invention also have an anti-microbial property, the composition of the invention are also effective to combat plaque and caries.

Particular embodiments according to the present invention will further be illustrated by way of Example.

### EXAMPLE 1

The following oral composition comprises two formulations: a PAP-gel formulation and a pH-adjusting formulation.

| Ingredient | Amount (% w/w) |
|---|---|
| PAP | 0.6 |
| Gantrez ® | 3.0 |
| Xanthan | 0.2 |
| Silica | 9.0 |
| Sodium hydroxide | 0.5 |
| Water | to 100 |

PAP is commercially available as Eureco HC-17 (ex. Ausimont).

This formulation has a pH of 4.

pH-adjusting formulation

| Ingredient | amount % (w/w) |
|---|---|
| Gantrez ® | 3.0 |
| Xanthan | 0.2 |
| Silica | 6.0 |
| Flavour | 1.0 |
| Saccharin | 0.25 |
| pH agent (sodium | to pH 11 |
| tripolyphosphate or NaOH) | |
| Potassium nitrate | 5.0 |
| Fluoride source | to 1500 ppm |
| Colour | as appropriate |
| Water | to 100 |

When both the PAP-formulation and the pH-sdjusting formulation are mixed together the pH of the resulting composition is between about 6.5 and 8.5.

1. An oral composition with an improved teeth whitening effect, comprising a first formulation comprising an effective amount of a peroxy amido phthalamide having the structural formula: 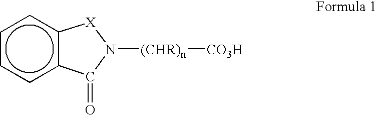
in which R is hydrogen or C₁-C₄ alkyl; n is 1 to 5; and X is C═O or SO₂,
wherein said first formulation has a pH of from 2 to 6
and a second formulation formulated such that when said second formulation is mixed with the first formulation the pH of the mixture is from 6.5 to 8.5.

2. Oral composition according to claim 1, wherein the second formulation has a pH ranging from 9 and 12.

3. Oral composition according to claim 1, wherein the pH of the mixture is from 7 to 8.

4. Oral composition according to claim 1, wherein the peroxy amido phthalamide is PAP.

5. A method of whitening the teeth, said method comprising the steps of mixing a first formulation comprising an effective amount of a peroxy amido phthalamide having the structural formula: 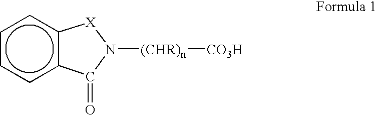
in which R is hydrogen or C₁-C₄ alkyl; n is 1 to 5; and X is C═O or SO₂,
wherein said first formulation has a pH of from 2 to 6
with a second formulation formulated such that when said second formulation is mixed with the first formulation the pH of the mixture is from 6.5 to 8.5 and then contacting said mixture with the teeth for a predetermined duration before rinsing.
